(19) United States
(12) Patent Application Publication
(10) Pub. No.: US 2008/0161666 A1
(43) Pub. Date: Jul. 3, 2008

(54) ANALYTE DEVICES AND METHODS

(75) Inventors: Benjamin J. Feldman, Oakland, CA (US); Gary Hayter, Oakland, CA (US); John C. Mazza, Pleasanton, CA (US); Andrew H. Naegeli, Walnut Creek, CA (US); Thomas A. Peyser, Menlo Park, CA (US); Marc B. Taub, Mountain View, CA (US)

Correspondence Address: JACKSON & CO., LLP, 6114 LA SALLE AVENUE, #507, OAKLAND, CA, 94611-2802, US

(73) Assignee: ABBOTT DIABETES CARE, INC., Alameda, CA (US)

(21) Appl. No.: 11/618,706

(22) Filed: Dec. 29, 2006

(51) Int. Cl. A61B 5/1495

(52) U.S. Cl. 600/365

(57) ABSTRACT

Analyte monitoring devices and methods are provided. Embodiments include devices and methods to evaluate the suitability of calibration periods of time.

### BACKGROUND OF THE INVENTION

The monitoring of the level of an analyte such as glucose or other analyte, such as lactate or oxygen, in certain individuals is vitally important to their health. For example, high or low levels of glucose or other analytes may have detrimental effects. The monitoring of glucose, for example, is particularly important to individuals with diabetes, as they must determine when corrective action is required, such as the administration of insulin to reduce glucose levels in their bodies or when additional glucose is needed to raise the level of glucose in their bodies.

A conventional technique used by many diabetics for personally monitoring their blood glucose level includes the periodic drawing of blood, the application of that blood to a test strip, and the determination of the blood glucose level using calorimetric, electrochemical, or photometric detection. This technique does not permit continuous or automatic monitoring of glucose levels in the body, but typically must be performed manually on a periodic basis. Unfortunately, the consistency with which the level of glucose is checked varies widely among individuals. Many diabetics find the periodic testing inconvenient and they sometimes forget to test their glucose level or do not have time for a proper test. In addition, some individuals wish to avoid the pain associated with the test. These situations may result in hyperglycemic or hypoglycemic episodes. An in vivo glucose sensor that continuously or automatically monitors the individual's glucose level enables individuals to more easily monitor their glucose, or other analyte, levels.

Devices have been developed for continuous or automatic monitoring of analytes, such as glucose, in the blood stream or interstitial fluid. Such devices include electrochemical sensors, at least a portion of which are operably positioned in contact with a body fluid, e.g., in a blood vessel or in the subcutaneous tissue of a patient.

As interest in analyte monitoring continues, there is interest in continuous analyte monitoring protocols that accurately monitor at least one analyte of an individual.

### SUMMARY OF THE INVENTION

Embodiments of the present invention relate to calibration of analyte monitoring devices and methods of calibration. Embodiments include devices and methods to optimize a calibration schedule.

Certain embodiments include calibration criteria. Calibration criteria may include analyte concentration and/or analyte rate of change.

Also provided are systems and kits.

### BRIEF DESCRIPTION OF THE DRAWINGS

The invention may be more completely understood in consideration of the following detailed description of various embodiments of the invention in connection with the accompanying drawings, in which:

FIG. 1 shows a block diagram of an exemplary embodiment of an analyte monitoring system;

FIG. 2 is a top view of an exemplary embodiment of an analyte sensor;

FIG. 3A is a cross-sectional view of the analyte sensor of FIG. 2;

FIG. 3B is a cross-sectional view of another embodiment of an analyte sensor;

FIG. 4A is a cross-sectional view of another embodiment of an analyte sensor;

FIG. 4B is a cross-sectional view of another embodiment of an analyte sensor;

FIG. 5 is a cross-sectional view of another embodiment of an analyte sensor;

FIG. 6 is an expanded top view of a tip-portion of an analyte sensor of one embodiment;

FIG. 7 is an expanded bottom view of a tip-portion of the analyte sensor of FIG. 6;

FIG. 8 is a side view of the analyte sensor of FIG. 2;

FIG. 9 is a cross-sectional view of an embodiment of an on-skin sensor control unit;

FIG. 10 is a block diagram of one embodiment of a receiver/display unit, according to the invention.

### DETAILED DESCRIPTION

Before the present invention is described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described.

It must be noted that as used herein and in the appended claims, the singular forms “a”, “an”, and “the” include plural referents unless the context clearly dictates otherwise. Any recited method can be carried out in the order of events recited or in any other order which is logically possible.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention.

The figures shown herein are not necessarily drawn to scale, with some components and features being exaggerated for clarity.

Embodiments are described primarily with respect to glucose systems and methods for convenience only and is in no way intended to limit the scope of the invention. It is understood that other analytes, analyte systems and methods may be employed.

Implantable analyte (e.g., glucose) sensors measure analyte in bodily fluid. For example, transcutaneously positioned glucose sensors may measure glucose in the interstitial fluid, rather than measuring glucose in externally expressed blood. One common type of glucose sensor is based on an electrochemical reaction which produces an electrical current proportional to the local glucose concentration in the tissue into which the sensor is placed. In many implantable systems, a reference measurement of glucose in externally expressed blood is employed to determine the constant of proportionality relating the sensor current to body glucose concentration (calibration). Under conditions of steady-state glucose values, a blood glucose measurement may be applied to determine the relevant constant of proportionality of the sensor without any additional acceptance constraints. The constant of proportionality may be described as the sensor sensitivity, namely, the proportional change in the sensor output current as a function of a corresponding change in the glucose concentration.

Glucose values in the body, however, are often not in steady-state conditions. This is especially true of patients with diabetes in whom the body's regulation of glycemia has been impaired by their disease. As a result, patients with diabetes often exhibit a high degree of temporal glycemic variability. This temporal variability can lead to errors in calibration of implanted, such as transcutaneously-inserted, glucose sensors for one or more of the following reasons:

### i) Physiological Lag

During high rates of change of an analyte such as glucose, the instantaneous glucose value in the blood may be different than the instantaneous value in other physiological compartments of the body such as in the interstitial fluid. During high rates of change of blood glucose, the physiological lag between two compartments can lead to discrepancies between measured glucose values made in the two compartments.

### ii) Sensor Membrane Lag

In order for glucose sensors to have a linear output response to analyte concentration such as glucose concentration, the flux of glucose to the transduction element in the sensor must be limited. An external membrane may be employed for this purpose. The use of a flux-limiting membrane on the sensor introduces an additional source of lag between the blood glucose and the measured glucose in the interstitial fluid during high rates of change of glucose.

iii) Non-Concurrent Sampling Lag

The temporal variability of glucose in both the blood and interstitial fluid requires that both compartments be sampled concurrently so that the transcutaneously-inserted sensor can be appropriately calibrated. If the two samples are not taken concurrently, the delay between the two measurements can introduce a source of error for the calibration which is exacerbated by high rates of change.

Embodiments include in vivo analyte monitoring systems, such as transcutaneously-inserted sensor systems, configured to evaluate periods of time for suitability to calibrate the system. One or more calibration criteria may be evaluated prior to evaluating whether calibration is suitable. Embodiments include systems that are capable of evaluating criteria to determine suitability of calibration. A variety of calibration criteria may be employed. In certain embodiments, a system may be configured (e.g., include an algorithm) to evaluate whether an analyte of interest is within a predetermined concentration range—a calibration-ready concentration range.

The exact limits of acceptable analyte concentration depend at least on the analyte of interest. For example, in certain embodiments the concentration range acceptable for calibration may range from about 60 to about 300 mg/dL.

Other criteria may be employed in addition to or in place of analyte concentration range criteria. For example, the rate of change of an analyte may fluctuate. This is true of, for example, glucose. Embodiments include systems that are configured to (e.g., include an algorithm) evaluate rates of change of an analyte. Evaluation may include whether the rate of change is within a predetermined rate of change—a calibration-ready rates of change. The system may be configured to perform such evaluation(s) prior to calibration of the transcutaneously-inserted sensor to permit calibration to times only when the rate of change (and/or other calibration criteria) is determined to be appropriate, e.g., is capable of restricting/preventing/permitting/determining the calibration according to predetermined criteria such as predetermined rates of change of the analyte. The limitation of acceptable calibration inputs to periods in which the rate of change is acceptable results in a higher level of accuracy and performance of a glucose sensor. Accordingly, embodiments provide a strict limit on the acceptability of calibration inputs to times in which the rate of change of the analyte of interest is within a prescribed range of acceptable values.

The exact limits of acceptable rates of change depend at least on the analyte of interest, and may include balancing the conflicting objectives of reducing the error associated with the rate of change and increasing the frequency of successful calibrations by the user. In certain glucose monitoring embodiments, the limits of acceptability for the absolute rate of change are small, e.g., less than about two milligrams per deciliter per minute. Such embodiments contemplate that many diabetics exhibited rates of change of glucose less than two milligrams per deciliter per minute as much as 75-805% of the time or more. The limits of acceptability also contemplate error associated with calibrating the sensor at rates of change. Other limits may of course be employed depending on the specifics of the application.

Based on numerical simulation of the error associated with high rates of change, rates of change in excess of about plus or minus 2 milligrams per deciliter per minute with respect to continuous glucose monitoring was determined to potentially result in significant decreases in accuracy relative to blood samples. Calibration during high rates of change can introduce systematic errors into continuous glucose monitor data for many hours. For example, if the physiological lag between an in vivo sensor measuring glucose in the interstitial fluid and an in vitro blood glucose measurement is assumed to be characterized by a time constant of about seven minutes, the apparent lag may be as high as about twenty minutes between the two data sets. If in addition the underlying blood glucose rises at a rate of about 3 milligrams per deciliter per minute, the effect of calibrating the sensor at such a time might be to introduce about a 60 milligram per deciliter offset into the continuous glucose monitor data. Accordingly, the application of an acceptance criteria for calibration based on the rate of change can result in significant improvements in the overall accuracy of the continuous glucose monitor.

Criteria (concentration and/or rate of change and/or other parameters) may be sampled for calibration suitability over a period of time, and for example, may be sampled over a time period of about 5 minutes or less, e.g., over about 2 minute or less, e.g., over about 1 minute or less, e.g., over about 45 seconds or less. Data may be used in raw form or processed form, e.g., may be averaged or the like. In other embodiments, a single point in time may be used, either in raw or processed form.

Embodiments may include systems that are configured to notify a user (audibly, visually, and/or in tactile manner) of evaluation results and/or suitable calibration periods of time. Whether or not the rate of change of an analyte and/or analyte concentration is/are suitable for calibration may be expressed to a user audibly, visually or in tactile manner (e.g., using vibratory indications). For example, a continuous analyte monitoring system may include a module with a user interface such as a display and/or speakers that may be configured to notify a user of suitability.

A system may be configured to determine a rate of change in any suitable manner. In many instances, this will be determined by the system as it monitors the analyte at least prior to an expected calibration event. In certain embodiments, the rate of change may be estimated, e.g., based at least in part on statistical sampling. A system may estimate a rate of change in anticipation of the system's first calibration, which first calibration event follows implantation of the sensor and initiation of the system by a user. For example, an initial calibration of a sensor may occur prior to the sensor sensitivity determination by a blood glucose measurement. In the absence of reference glucose measurements from externally expressed blood, a pre-determined sensor sensitivity derived from the sensor sensitivities of a statistical sampling of a given sensor population, such as a given manufacturing lot or the like, may be used to determine the rate of change of glucose. This sensitivity value may be provided to the user in any suitable manner, e.g., as a sensor calibration code or the like, which may then be input into the device by the user or otherwise entered into the system without user action (e.g., a calibration reader, etc.). Alternatively or in addition to, manufacturing and production tolerances may be established such that a given population of sensors (e.g., a manufacturing lot) has the same sensitivity within a small margin. In this case, the sensor sensitivity may be provided by the manufacturing tolerances and provided either as input to the sensor system or incorporated directly into the sensor system operating software. After the initial calibration, the rate of change of glucose may be estimated from the calibrated glucose signal, the pre-determined factory sensor sensitivity or a combination of both.

In certain glucose monitoring system embodiments, the calibration criteria may include a concentration range acceptable for calibration of about 60 to about 300 mg/dL and a rate of change of glucose of about 2 mg/dL/min. The system may not display or otherwise present glucose values of the system until calibration criteria is met and a first calibration event is performed, i.e., until the system is calibrated.

Embodiments may also include methods to determine a suitable period of time to calibrate an analyte system. Certain embodiments include methods to evaluate calibration criteria such as for example analyte concentrations and/or the rate of change of an analyte, and the like, and may include accepting and/or rejecting and/or permitting and/or restricting calibration events based at least in part on calibration evaluation, such as for example the determined rate of change of the analyte. Certain embodiments include methods to optimize a calibration schedule of an analyte monitoring system.

Certain embodiments include determining the rate of change of glucose prior to establishing the constant of proportionality relating the blood glucose to the interstitial glucose. Embodiments may include comparing the determined rate of change to predetermined acceptance rate of change criteria (e.g., when the rate of change is small such as when below about plus or minus 2 milligrams per deciliter per minute, or other suitable value). A constant of proportionality relating the blood glucose to the interstitial glucose may be determined or accepted if the rate of change meets the acceptance criteria or will not determined or accepted if the rate of change does not meet the acceptance criteria, i.e., the rate of change is rejected. In certain embodiments, if the rate of change is rejected, the process may be repeated one or more times until a suitable rate of change is determined. Calibration of the sensor may then be initiated. Certain embodiments include systems configured to be calibrated by single point calibration, as described for example in one or more of U.S. Pat. Nos. 5,965,380, 6,083,710, 6,121,009, 6,162,611, 6,284,478, 5,514,718, 5,262,305.

As noted above, the rate of analyte change may be determined in any suitable manner, including those described above. In certain continuous monitoring systems and methods, one or more calibrations are needed after initialization of the system by a user. In certain embodiments, a period of time may be required between initialization of the system and a first calibration. The period of time may be predetermined or may be determined at least in part according to embodiments of the invention, e.g., may be determined at least in part based on calibration criteria, e.g., by the determination of a suitable analyte rate of change and/or analyte concentration.

As noted above, an initial or first calibration of a sensor occurs prior to the sensor sensitivity determination by a blood glucose measurement. Accordingly, embodiments of the subject methods include determining and/or providing to a user for input to the system (or directly to a system) a pre-determined sensor sensitivity derived from the sensor sensitivities of a statistical sampling of a sensor lot to determine the rate of change of glucose. Alternative embodiments include establishing and/or providing to a user (or directly to a system) manufacturing and production tolerances such that all sensors of a given sensor population are assigned the same sensitivity within a small margin. The sensor sensitivity may thus be attributed to manufacturing tolerances and provided either as input to the sensor system or incorporated directly into the sensor system operating software. After the initial calibration, the rate of change of glucose may be estimated from the calibrated glucose signal, the pre-determined factory sensor sensitivity or a combination of both.

Accordingly, embodiments include multiple calibration events and prior to at least one of the events, the rate of analyte change is observed and determined to be calibration acceptable or not based on predetermined criteria.

Certain embodiments include initiating an implantable analyte monitoring system and continually or periodically evaluating calibration criteria to determine a suitable time for calibrations based at least in part on the evaluated criteria. Accordingly, dynamic calibration systems are contemplated, as well as methods to dynamically calibrate a system. Once criteria is satisfied (for example the analyte concentration and/or analyte rate of change), calibration of the sensor may begin and may include providing a reference measurement such as by a calibration code or externally expressed blood for additional calibrations. In many embodiments, criteria for initial calibration is evaluated (and typically determined to be satisfied) within less than about 24 hours after initialization, e.g., less than about 15 hours, e.g., less than about 10 hours, e.g., less than about 5 hours. The process may be repeated for additional calibration events. Certain embodiments include calibrating a system by single point calibration.

Embodiments of the subject invention may be manually implemented, e.g., by a continuous analyte monitoring system user and/or a healthcare provider thereof, or may be fully or at least partially automated, e.g., by a system's processing system. Processors may be employed, e.g., that implement aspects of embodiments. Instructions for carrying embodiments of the invention may be embodied on a computer readable medium, where such medium may be readable by the system which includes hardware and software for carrying out the instructions. The processors may be included in a continuous monitoring system or otherwise couple able thereto. For example, a module that determines glucose values and notifies a user of the values may be employed. Such as module may also include component for determining a reference measurement in externally expressed blood, e.g., applied to a test strip and received by the module (for example received by a strip port of the module) for analyte determination. Such a module may include a transmitter, receiver, transceiver, personal computer, PDA, cell phone, or the like. Non limiting examples of representative analyte systems are described below. Exemplary analyte systems that may be employed are described in, for example, U.S. Pat. Nos. 6,134,461, 6,175,752, 6,121,611, 6,560,471, 6,746,582, and elsewhere, the disclosures of which are herein incorporated by reference.

Exemplary Analyte Monitoring Systems

As described above, embodiments of the present invention may include analyte sensor devices and methods and are applicable to analyte monitoring systems and methods that employ an analyte sensor—at least a portion of which is positionable beneath the skin of the user for the in vivo determination of a concentration of an analyte, such as glucose, lactate, and the like, in a body fluid. The sensor may be, for example, subcutaneously positionable in a patient for the continuous or periodic monitoring an analyte in a patient's interstitial fluid. This may be used to derive the glucose level in the patient's bloodstream. The sensors of the subject invention also include in vivo analyte sensors insertable into a vein, artery, or other portion of the body containing fluid. A sensor may be configured for monitoring the level of the analyte over a time period which may range from minutes, hours, days, weeks, or longer. Of interest are analyte sensors, such as glucose sensors, that are capable of providing analyte data for about one hour or more, e.g., about a few hours or more, e.g., about a few days of more, e.g., about three or more days, e.g., about five days or more, e.g., about seven days or more, e.g., about several weeks or months.

The particular configuration of a sensor and other units used in an analyte monitoring system may depend on the use for which the sensor and system are intended and the conditions under which the sensor and system will operate. As noted above, embodiments include a sensor configured for implantation into a patient or user. The term “implantation” is meant broadly to include wholly implantable sensors as well as sensors in which only a portion of which is implantable under the skin and a portion of which resides above the skin, e.g., for contact to a transmitter, receiver, transceiver, processor, etc. For example, implantation of the sensor may be made in the arterial or venous systems for direct testing of analyte levels in blood. Alternatively, a sensor may be implanted in the interstitial tissue for determining the analyte level in interstitial fluid. This level may be correlated and/or converted to analyte levels in blood or other fluids. The site and depth of implantation may affect the particular shape, components, and configuration of the sensor. Subcutaneous implantation may be desired, in some cases, to limit the depth of implantation of the sensor. Sensors may also be implanted in other regions of the body to determine analyte levels in other fluids.

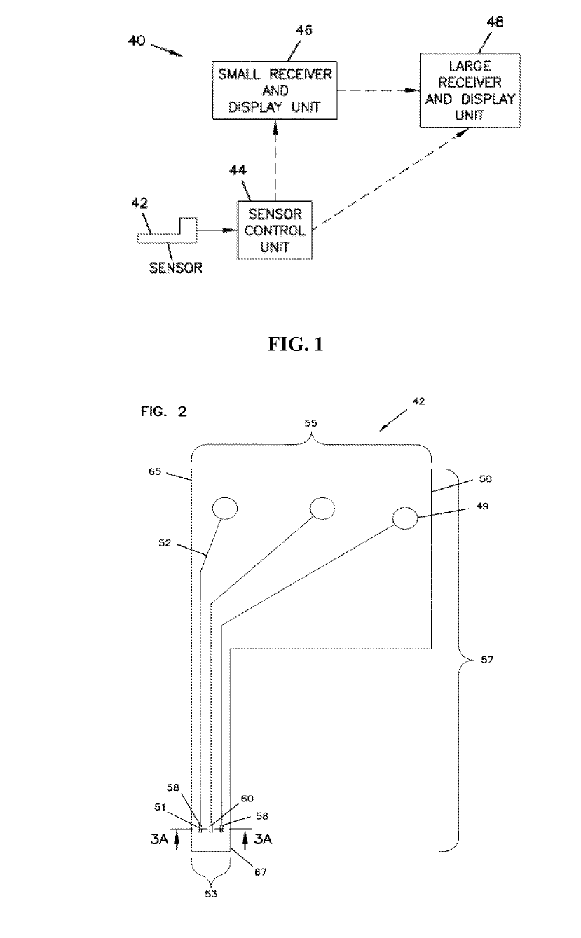

An exemplary embodiment of an analyte monitoring system 40 including implantable analyte sensor 42 is illustrated in block diagram form in FIG. 1. The analyte monitoring system 40 includes, at minimum, a sensor 42, at least a portion of the sensor which is configured for implantation (e.g., subcutaneous, venous, or arterial implantation) into a patient, and a sensor control unit 44. The sensor 42 is coupleable to the sensor control unit 44 which is typically attachable or otherwise held in place to the skin of a patient. The sensor control unit 44 operates the sensor 42, including, for example, providing a voltage across the electrodes of the sensor 42 and collecting signals from the sensor 42.

The sensor control unit 44 may evaluate the signals from the sensor 42 and/or transmit the signals to one or more optional receiver/display units 46, 48 for evaluation. The sensor control unit 44 and/or the receiver/display units 46, 48 may display or otherwise communicate the current level of the analyte. Furthermore, the sensor control unit 44 and/or the receiver/display units 46, 48 may indicate to the patient, via, for example, an audible, visual, or other sensory-stimulating alarm, when the level of the analyte is at or near a threshold level and/or information about calibration such as suitability to calibrate the system. Alarms may be included. For example if glucose is monitored, an alarm may be used to alert the patient to a hypoglycemic or hyperglycemic glucose level and/or to impending hypoglycemia or hyperglycemia.

A sensor 42 includes at least one working electrode 58 and a substrate 50, as shown in FIG. 2. The shape of sensor 42 is for exemplary purposes only. It is understood that any other shapes are contemplated. The sensor 42 may also include at least one counter electrode 60 (or counter/reference electrode) and/or at least one reference electrode 62 (see for example FIG. 7). The counter electrode 60 and/or reference electrode 62 may be formed on the substrate 50 or may be separate units. The working electrode or electrodes 58 are formed using conductive materials 52. The counter electrode 60 and/or reference electrode 62, as well as other optional portions of the sensor 42, such as an optional temperature probe 66 (see for example FIG. 7), may also be formed using conductive material 52. The conductive material 52 may be formed over a smooth surface of the substrate 50 or within channels 54 formed by, for example, embossing, indenting or otherwise creating a depression in the substrate 50.

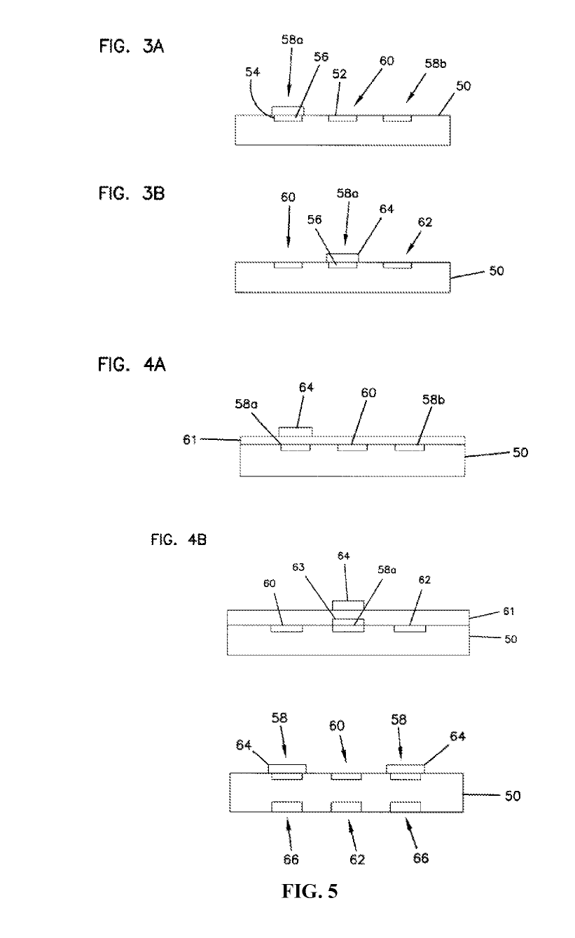

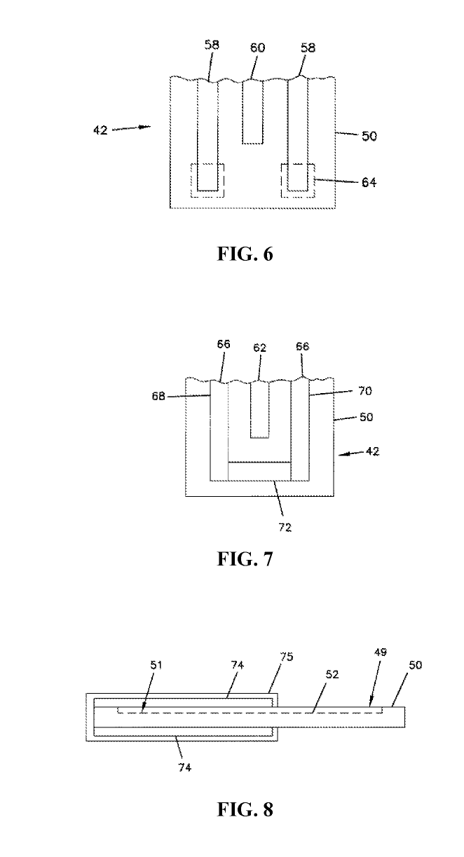

A sensing layer 64 (see for example FIGS. 3B, 4A-4B, 5 and 6) may be provided proximate to or on at least one of the working electrodes 58 to facilitate the electrochemical detection of the analyte and the determination of its level in the sample fluid, particularly if the analyte can not be electrolyzed at a desired rate and/or with a desired specificity on a bare electrode.

In addition to the electrodes 58, 60, 62 and the sensing layer 64, the sensor 42 may also include optional components such as one or more of the following: a temperature probe 66 (see for example FIGS. 5 and 7), a mass transport limiting layer 74, e.g., a matrix such as a membrane or the like, (see for example FIG. 8), a biocompatible layer 75 (see for example FIG. 8), and/or other optional components, as described below.

The substrate 50 may be formed using a variety of non-conducting materials, including, for example, polymeric or plastic materials and ceramic materials. Suitable materials for a particular sensor 42 may be determined, at least in part, based on the desired use of the sensor 42 and properties of the materials.

In addition to considerations regarding flexibility, it is often desirable that a sensor 42 should have a substrate 50 which is non-toxic. Although the substrate 50 in at least some embodiments has uniform dimensions along the entire length of the sensor 42, in other embodiments, the substrate 50 has a distal end 67 and a proximal end 65 with different widths 53, 55, respectively, as illustrated in FIG. 2.

At least one conductive trace 52 may be formed on the substrate for use in constructing a working electrode 58. In addition, other conductive traces 52 may be formed on the substrate 50 for use as electrodes (e.g., additional working electrodes, as well as counter, counter/reference, and/or reference electrodes) and other components, such as a temperature probe. The conductive traces 52 may extend most of the distance along a length 57 of the sensor 50, as illustrated in FIG. 2, although this is not necessary. The placement of the conductive traces 52 may depend on the particular configuration of the analyte monitoring system (e.g., the placement of control unit contacts and/or the sample chamber in relation to the sensor 42). For implantable sensors, particularly subcutaneously implantable sensors, the conductive traces may extend close to the tip of the sensor 42 to minimize the amount of the sensor that must be implanted.

The conductive traces may be formed using a conductive material 56 such as carbon (e.g., graphite), a conductive polymer, a metal or alloy (e.g., gold or gold alloy), or a metallic compound (e.g., ruthenium dioxide or titanium dioxide), and the like. Conductive traces 52 (and channels 54, if used) may be formed with relatively narrow widths. In embodiments with two or more conductive traces 52 on the same side of the substrate 50, the conductive traces 52 are separated by distances sufficient to prevent conduction between the conductive traces 52. The working electrode 58 and the counter electrode 60 (if a separate reference electrode is used) may be made using a conductive material 56, such as carbon.

The reference electrode 62 and/or counter/reference electrode may be formed using conductive material 56 that is a suitable reference material, for example silver/silver chloride or a non-leachable redox couple bound to a conductive material, for example, a carbon-bound redox couple.

The electrical contact 49 may be made using the same material as the conductive material 56 of the conductive traces 52, or alternatively, may be made from a carbon or other non-metallic material, such as a conducting polymer.

A number of exemplary electrode configurations are described, however, is understood that other configurations may also be used. In certain embodiments, e.g., illustrated in FIG. 3A, the sensor 42 includes two working electrodes 58a, 58b and one counter electrode 60, which also functions as a reference electrode. In another embodiment, the sensor includes one working electrode 58a, one counter electrode 60, and one reference electrode 62, as shown for example in FIG. 3B. Each of these embodiments is illustrated with all of the electrodes formed on the same side of the substrate 50. Alternatively, one or more of the electrodes may be formed on an opposing side of the substrate 50.

Some analytes, such as oxygen, may be directly electrooxidized or electroreduced on the working electrode 58. Other analytes, such as glucose and lactate, require the presence of at least one electron transfer agent and/or at least one catalyst to facilitate the electrooxidation or electroreduction of the analyte. Catalysts may also be used for those analyte, such as oxygen, that can be directly electrooxidized or electroreduced on the working electrode 58. For these analytes, each working electrode 58 has a sensing layer 64 formed proximate to or on a working surface of the working electrode 58. In many embodiments, the sensing layer 64 is formed near or on only a small portion of the working electrode 58, e.g., nears a tip of the sensor 42. The sensing layer 64 includes one or more components designed to facilitate the electrolysis of the analyte. The sensing layer 64 may be formed as a solid composition of the desired components (e.g., an electron transfer agent and/or a catalyst). The sensing layer 64 may also include a catalyst which is capable of catalyzing a reaction of the analyte. The catalyst may also, in some embodiments, act as an electron transfer agent.

To electrolyze the analyte, a potential (versus a reference potential) may be applied across the working and counter electrodes 58, 60. When a potential is applied between the working electrode 58 and the counter electrode 60, an electrical current will flow.

Those skilled in the art will recognize that there are many different reactions that will achieve the same result; namely the electrolysis of an analyte or a compound whose level depends on the level of the analyte.

A variety of optional items may be included in the sensor. One optional item is a temperature probe 66 (see for example FIG. 7).

The sensors of the subject invention are biocompatible. Biocompatibility may be achieved in a number of different manners. For example, an optional biocompatible layer 74 may be formed over at least that portion of the sensor 42 which is inserted into the patient, as shown in FIG. 8.

An interferant-eliminating layer (not shown) may be included in the sensor 42. The interferant-eliminating layer may include ionic components, such as Nafion® or the like, incorporated into a polymeric matrix to reduce the permeability of the interferant-eliminating layer to ionic interferants having the same charge as the ionic components.

A mass transport limiting layer 74 may be included with the sensor to act as a diffusion-limiting barrier to reduce the rate of mass transport of the analyte, for example, glucose or lactate, into the region around the working electrodes 58. Exemplary layers that may be used are described for example, in U.S. Pat. No. 6,881,551, and elsewhere.

Some or all of the layers described herein may be provided as integrated, e.g., a single layer, or may be discrete layers.

A sensor of the various embodiments of the subject invention may be adapted to be a replaceable component in an in vivo analyte monitor, and particularly in an implantable analyte monitor. As described above, in many embodiments the sensor is capable of operation over a period of days or more, e.g., a period of operation may be at least about one day, e.g., at least about three days, e.g., at least about five days, e.g., at least about one week or more, e.g., one month or more. The sensor may then be removed and replaced with a new sensor.

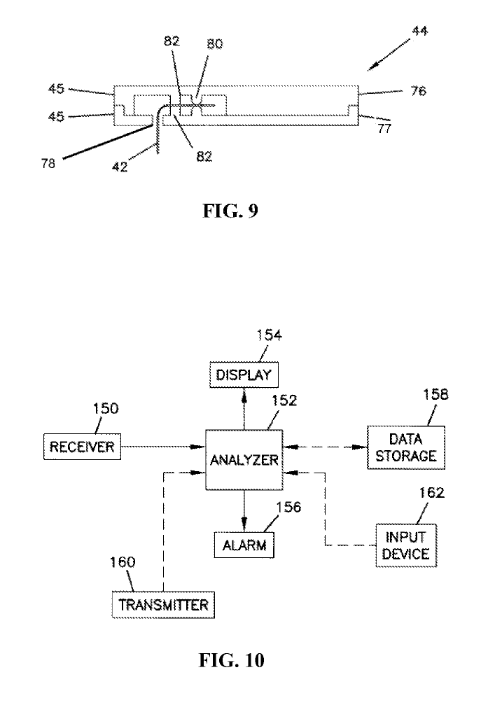

Referring back to FIG. 1, the sensor control unit 44 may be configured to be placed on the skin of a patient. One embodiment of the on-skin sensor control unit 44 is shaped to enhance concealment. However, it may be shaped otherwise. The on-skin sensor control unit 44 includes a housing 45 (see for example, FIG. 9). In some embodiments, the housing 45 of the on-skin sensor control unit 44 is a single piece. The conductive contacts 80 may be formed on the exterior of the housing 45 or on the interior of the housing 45 provided there is a port 78 in the housing 45 through which the sensor 42 can be directed to access the conductive contacts 80. In other embodiments, the housing 45 of the on-skin sensor control unit 44 is formed in at least two separate portions that fit together to form the housing. Alternatively, at least some of the two or more portions of the housing 45 may be connected together, for example, by a hinge or the like, to facilitate the coupling of the portions to form the housing 45 of the on-skin sensor control unit 44.

The on-skin sensor control unit 44 is typically attachable to the skin of the patient. For example, the housing 45 of the on-skin sensor control unit 44 may be attachable to the skin using a mounting unit 77. A mounting unit 77 may be integral with the control unit 44 or may be separable therefrom. The sensor 42 and the electronic components within the on-skin sensor control unit 44 are coupled via conductive contacts 80.

The on-skin sensor control unit 44 may include at least a portion of the electronic components that operate the sensor 42 and the analyte monitoring device system 40. The electronic components of the on-skin sensor control unit 44 may include a power supply to operate the on-skin control unit 44 and the sensor 42, a sensor circuit to obtain signals from and operating the sensor, a measurement circuit to convert sensor signals to a desired format, and a processing circuit to, at minimum, obtain signals from the sensor circuit and/or measurement circuit and provide the signals to an optional transmitter. In some embodiments, a processing circuit may also partially or completely evaluate the signals from the sensor and convey the resulting data to an optional transmitter and/or activate an optional alarm system if the analyte level exceeds a threshold. The processing circuit may include digital logic circuitry.

The on-skin sensor control unit 44 may optionally contain a transmitter or transceiver for transmitting the sensor signals or processed data from the processing circuit to a receiver (or transceiver)/display unit; a data storage unit for temporarily or permanently storing data from the processing circuit; a temperature probe circuit for receiving signals from and operating a temperature probe a reference voltage generator for providing a reference voltage for comparison with sensor-generated signals; and/or a watchdog circuit that monitors the operation of the electronic components in the on-skin sensor control unit 44.

In certain embodiments, an on-skin control unit 44 may include optional components such as a receiver (or transceiver) to receive, for example, calibration data; a calibration storage unit to hold, for example, factory-set calibration data, calibration data obtained via a receiver and/or operational signals received, for example, from a receiver/display unit or other external device; an alarm system for warning the patient; and a deactivation switch, for example to turn off the alarm system.

In certain embodiments, the data (e.g., a current signal, a converted voltage or frequency signal, or fully or partially analyzed data) from the control unit processing circuit is transmitted to one or more receiver/display units using a transmitter in the on-skin sensor control unit 44. The transmitter may include an antenna, such as a wire or similar conductor, formed in the housing.

In addition to a transmitter, an optional receiver may be included in the on-skin sensor control unit 44. In some cases, the transmitter is a transceiver, operating as both a transmitter and a receiver. The receiver (and/or receiver display/units 46, 48) may be used to receive calibration data for the sensor 42. The calibration data may be used by the processing circuit to correct signals from the sensor 42. This calibration data may be transmitted by the receiver/display unit 46, 48 or from some other source such as a control unit in a doctor's office.

The on-skin sensor control unit 44 may include an optional data storage unit which may be used to hold data (e.g., measurements from the sensor or processed data).

In some embodiments, the analyte monitoring device 40 includes only an on-skin control unit 44 and a sensor 42. In some embodiments, the analyte monitoring device 40 includes only an on-skin control unit 44 and a sensor 42 and a receiver (46 or 48).

One or more receiver/display units 46, 48 may be provided with the analyte monitoring device 40 for easy access to the data generated by the sensor 42 and may, in some embodiments, process the signals from the on-skin sensor control unit 44 to determine the concentration or level of analyte in the subcutaneous tissue. The receiver may be a transceiver. Receivers may be palm-sized and/or may be adapted to fit on a belt or within a bag or purse that the patient carries.

The receiver/display units 46, 48 (either or both receiver/display units), as illustrated in block form at FIG. 10, may include a receiver 150 to receive data from the on-skin sensor control unit 44, an analyzer 152 to evaluate the data, a display 154 to provide information to the patient, and an alarm system 156, e.g., to warn the patient when a condition arises. The receiver/display units 46, 48 may also optionally include a data storage device 158, a transmitter 160, and/or an input device 162. Analyzer 152 may be configured to analyze calibration criteria. Analyzer 152 may determine suitability of a period of time for calibration.

Data received by the receiver 150 may be forwarded to an analyzer 152. The output from the analyzer 152 may be provided to a display 154. The receiver/display units 46, 48 may also include a number of optional items such as a data storage unit 158 store data, a transmitter 160 which can be used to transmit data, and an input device 162, such as a keypad or keyboard.

In certain embodiments, the receiver/display unit 46, 48 (one or both) is integrated or otherwise coupleable with a calibration unit (not shown). For example, the receiver/display unit 46, 48 may, for example, include a conventional blood glucose monitor. Devices may be used including those that operate using, for example, electrochemical and calorimetric blood glucose assays, assays of interstitial or dermal fluid, and/or non-invasive optical assays. When a calibration of the implanted sensor is needed, the patient may use the integrated in vitro monitor to generate a reading. The reading may then, for example, automatically be sent by the transmitter 160 of the receiver/display unit 46, 48 to calibrate the sensor 42.

Integration with a Drug Administration System

The embodiments of the subject invention may also include sensors used in sensor-based drug delivery systems. The system may provide a drug to counteract the high or low level of the analyte in response to the signals from one or more sensors. Alternatively, the system may monitor the drug concentration to ensure that the drug remains within a desired therapeutic range. The drug delivery system may include one or more (e.g., two or more) sensors, an on-skin sensor control unit, a receiver/display unit, a data storage and controller module, and a drug administration system. In some cases, the receiver/display unit, data storage and controller module, and drug administration system may be integrated in a single unit. The sensor-based drug delivery system may use data from the one or more sensors to provide necessary input for a control algorithm/mechanism in the data storage and controller module to adjust the administration of drugs. As an example, a glucose sensor could be used to control and adjust the administration of insulin.

Kits

Finally, kits for use in practicing the subject invention are also provided. The subject kits may include one or more sensors as described herein. Embodiments may also include a sensor and/or a sensor positioning device and/or transmitter and/or receiver.

In addition to one or more of the above-described components, the subject kits may also include written instructions for using a sensor to obtain analyte information. The instructions may be printed on a substrate, such as paper or plastic, etc. As such, the instructions may be present in the kits as a package insert, in the labeling of the container of the kit or components thereof (i.e., associated with the packaging or sub-packaging) etc. In other embodiments, the instructions are present as an electronic storage data file present on a suitable computer readable storage medium, e.g., CD-ROM, diskette, etc. In yet other embodiments, the actual instructions are not present in the kit, but rather include directions to obtain the instructions from a remote source, e.g., via the Internet, are provided. An example of this embodiment is a kit that includes a web address where the instructions can be viewed and/or from which the instructions can be downloaded. As with the instructions, this means for obtaining the instructions is recorded on a suitable substrate.

In many embodiments of the subject kits, the components of the kit are packaged in a kit containment element to make a single, easily handled unit, where the kit containment element, e.g., box or analogous structure, may or may not be an airtight container, e.g., to further preserve the one or more sensors and additional reagents (e.g., control solutions), if present, until use.

An analyte monitoring system in one embodiment includes an analyte sensor, a processor coupled to the sensor to determine the concentration of analyte, and a user interface to present analyte information to a user, where the system is configured to evaluate calibration criteria.

The calibration criteria may include analyte concentration or analyte rate of change or analyte concentration and analyte rate of change, and further, where calibration criteria may include analyte concentration and analyte rate of change.

The analyte may include glucose.

The system may be configured to determine the suitability of the system for calibration based on whether glucose concentration is in the range from about 60 mg//dL and 300 mg/dL. Moreover, the system may be suitable if glucose concentration is in the range from about 60 mg//dL and 300 mg/dL.

Further, the system may be configured to determine the suitability of the system for calibration based on whether the glucose rate of change is less than about 2 mg/dL/minute.

In addition, the system may be suitable if the glucose rate of change is less than about 2 mg/dL/minute.

In one aspect, the system may be configured to prevent presentation of analyte concentration to a user until calibration criteria is satisfied, where the system may be configured to prevent presentation of analyte information to a user until the system is calibrated.

In a further aspect, the system may further include a calibration module.

The calibration module may include an analyte test strip reader.

The system may further include a control unit coupled to the sensor, where the control unit may include a transmitter or transceiver.

The system may further include a receiver to receive analyte information from the control unit.

In yet another aspect, the system may prevent calibration until calibration criteria acceptance.

A glucose monitoring system in accordance with another embodiment includes a glucose sensor, and an algorithm embodied on a computer readable medium to evaluate the suitability of calibration periods of time to calibrate the sensor.

The algorithm may evaluate calibration criteria, and further, where calibration criteria may include glucose concentration.

Further, the calibration criteria may include glucose rate of change.

The sensor may include a transcutaneous sensor.

Additionally, the system may be configured to restrict calibration events to periods of time of suitability.

A glucose monitoring system programmed to evaluate calibration criteria comprising suitable rates of change of glucose.

The rate of change of glucose may include whether the glucose rate of change is less than about 2 mg/dL/minute.

The criteria may be suitable if the glucose rate of change is less than about 2 mg/dL/minute.

The system may be configured to prevent presentation of analyte concentration information to a user the rate of change criteria is suitable.

The system may be programmed to prevent presentation of analyte information to a user until the system is calibrated.

In still another aspect, the system may further include a calibration module, where the calibration module may include an analyte test strip reader.

A method of calibrating an analyte monitoring system in accordance with yet another embodiment includes evaluating calibration criteria, determining the suitability of calibration periods of time based on the evaluated criteria, and calibrating the analyte monitoring system if a period of time is determined suitable for calibration.

The method may include not calibrating the system if a period of time is determined to be unsuitable.

In another aspect, the method may include evaluating analyte concentration or analyte rate of change or analyte concentration and analyte rate of change, where calibration criteria may include analyte concentration and analyte rate of change.

The analyte may include glucose.

The criteria may include determining whether the concentration ranges from about 60 mg//dL and 300 mg/dL.

In still another aspect, the criteria may include determining whether rate of change is less than about 2 mg/dL/minute.

The method may further include masking the presentation of analyte concentration to a user until calibration criteria is satisfied.

It is evident from the above results and discussion that the above-described invention provides devices and methods for continuous analyte monitoring. The above-described invention provides a number of advantages some of which are described herein and which include, but are not limited to, the ability to determine suitable periods of time to calibrate an analyte monitoring system. As such, the subject invention represents a significant contribution to the art.

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto.

What is claimed is:

1. An analyte monitoring system, the system comprising:
an analyte sensor;
a processor coupled to the sensor to determine the concentration of analyte; and
a user interface to present analyte information to a user;
wherein the system is configured to evaluate calibration criteria.

2. The analyte monitoring system of claim 1, wherein calibration criteria comprises analyte concentration or analyte rate of change or analyte concentration and analyte rate of change.

3. The analyte monitoring system of claim 2, wherein calibration criteria comprises analyte concentration and analyte rate of change.

4. The analyte monitoring system of claim 3, wherein the analyte is glucose.

5. The analyte monitoring system of claim 4, wherein the system is configured to determine the suitability of the system for calibration based on whether glucose concentration is in the range from about 60 mg//dL and 300 mg/dL.

6. The analyte monitoring system of claim 5, wherein the system is suitable if glucose concentration is in the range from about 60 mg//dL and 300 mg/dL.

7. The analyte monitoring system of claim 4, wherein the system is configured to determine the suitability of the system for calibration based on whether the glucose rate of change is less than about 2 mg/dL/minute.

8. The analyte monitoring system of claim 5, wherein the system is suitable if the glucose rate of change is less than about 2 mg/dL/minute.

9. The analyte monitoring system of claim 1, wherein the system is configured to prevent presentation of analyte concentration to a user until calibration criteria is satisfied.

10. The analyte monitoring system of claim 9, wherein the system is configured to prevent presentation of analyte information to a user until the system is calibrated.

11. The analyte monitoring system of claim 1, wherein the system further comprises a calibration module.

12. The analyte monitoring system of claim 1, wherein the calibration module is an analyte test strip reader.

13. The analyte monitoring system of claim 1, further comprising a control unit coupled to the sensor.

14. The analyte monitoring system of claim 13, wherein the control unit comprises a transmitter or transceiver.

15. The analyte monitoring system of claim 14, wherein the system comprises a receiver to receive analyte information from the control unit.

16. The analyte monitoring system of claim 1, wherein the system prevents calibration until calibration criteria acceptance.

17. A glucose monitoring system comprising:
a glucose sensor; and
an algorithm embodied on a computer readable medium to evaluate the suitability of calibration periods of time to calibrate the sensor.

18. The system of claim 17, wherein the algorithm evaluates calibration criteria.

19. The system of claim 18, wherein calibration criteria comprises glucose concentration.

20. The system of claim 18, wherein calibration criteria comprises glucose rate of change.

21. The system of claim 17, wherein the sensor is a transcutaneous sensor.

22. The system of claim 21, wherein the system is configured to restrict calibration events to periods of time of suitability.

23. A glucose monitoring system programmed to evaluate calibration criteria comprising suitable rates of change of glucose.

24. The system of claim 23, wherein the rate of change of glucose comprises whether the glucose rate of change is less than about 2 mg/dL/minute.

25. The system of claim 24, wherein the criteria is suitable if the glucose rate of change is less than about 2 mg/dL/minute.

26. The system of claim 25, wherein the system is configured to prevent presentation of analyte concentration information to a user the rate of change criteria is suitable.

27. The system of claim 25, wherein the system is programmed to prevent presentation of analyte information to a user until the system is calibrated.

28. The system of claim 23, wherein the system further comprises a calibration module.

29. The system of claim 28, wherein the calibration module is an analyte test strip reader.

30. A method of calibrating an analyte monitoring system, the method comprising:
evaluating calibration criteria;
determining the suitability of calibration periods of time based on the evaluated criteria; and
calibrating the analyte monitoring system if a period of time is determined suitable for calibration.

31. The method of claim 30, comprising not calibrating the system if a period of time is determined to be unsuitable.

32. The method of claim 30, wherein the method comprises evaluating analyte concentration or analyte rate of change or analyte concentration and analyte rate of change.

33. The method of claim 32, wherein calibration criteria comprises analyte concentration and analyte rate of change.

34. The method of claim 33, wherein the analyte is glucose.

35. The method of claim 33, wherein criteria comprises determining whether the concentration ranges from about 60 mg//dL and 300 mg/dL.

36. The method of claim 35, wherein criteria comprises determining whether rate of change is less than about 2 mg/dL/minute.

37. The method of claim 36, further comprising masking the presentation of analyte concentration to a user until calibration criteria is satisfied.
